(19) United States
(12) Patent Application Publication
(10) Pub. No.: US 2018/0055338 A1
(43) Pub. Date: Mar. 1, 2018

(54) Endoscope Ideal for Single Use

(71) Applicant: WISETEK INTERNATIONAL LLC, a California limited liability company, Fremont, CA (US)

(72) Inventors: XiBo Wei, Fremont, CA (US); GePing Liu, San Jose, CA (US); XiYi Wei, Fremont, CA (US)

(73) Assignee: WISETEK INTERNATIONAL LLC, a California limited liability company, Fremont, CA (US)

(21) Appl. No.: 15/790,914

(22) Filed: Oct. 23, 2017

Related U.S. Application Data
(63) Continuation-in-part of application No. 15/246,636, filed on Aug. 25, 2016.
Application No. 15/246,636 is a continuation-in-part of application No. 15/649,485, filed on Jul. 13, 2017.

(30) Foreign Application Priority Data
Nov. 24, 2016 (CN) 201611041752.8
Nov. 24, 2016 (CN) 201611041782.9

(51) Int. Cl. A61B 1/005; A61B 1/00

(52) U.S. Cl. CPC A61B 1/0055; A61B 1/0052; A61B 1/0011; A61B 1/00009; A61B 1/00165; A61B 1/00142; A61B 1/00062

(57) ABSTRACT

The present invention is about an endoscope ideal for a single use. The endoscope is able to enter human's body capturing real-time endourologic imaging, bending the distal end, and allowing the entrance of surgical tools. The distal end carries imaging sensor(s) and MicroLED/OLED/infrared LED/Laser LED/Laser diode/LED lighting source(s). The lever on the handle is able to control the distal end for bending up to 275 degrees in two directions, for rotating the distal end up to 360 degrees, and for adjusting an image sensor's orientation.

### RELATED APPLICATIONS

This application is a continuation-in-part (CIP) application of prior application Ser. No. 15/246,636 filed on 2016 Aug. 25.

This application is also a continuation-in-part (CIP) application of prior application Ser. No. 15/649,485 filed on 2017 Jul. 13 claiming a priority date as 2016 Nov. 2024 on which both Chinese Patent Applications No. 201611041752.8 and 201611041782.9 were filed.

Present application seeks the same priorities as that of these applications in part. The entire contents of each of which are incorporated herein by reference.

### BACKGROUND OF THE INVENTION

### 1. Field of the Invention

The present invention belongs to a field of biomedical instrumentation. More specifically the present invention belongs to a field of endoscopes including ureteroscopes. Specifications below use a ureteroscope in the present invention as an example. Similar concepts are easily extended to all types of endoscopes in general.

### 2. Description of the Related Art

A ureteroscopy is an examination of an upper urinary tract of a patient. A ureteroscope passes through a urethra and a bladder, and into a ureter. The lower ⅔ of a ureter can be accessed by this procedure. The ureteroscope is used in diagnosis and treatment of disorders such as kidney stones, which can be removed during a ureteroscopy. A flexible ureteroscope has an actively deflectable distal end, enabling deflection of the distal end to 170 degrees. A latest generation of flexible ureteroscope has taken deflection even further; one model incorporates an active secondary deflection mechanism, while another can deflect up to 270 degrees.

However, most ureteroscopes are not for single uses. It is time and labor consuming to sterilize the ureteroscope after uses. However, the risk of cross infection still exists. There are partially disposable ureteroscopes with removable distal ends, single-use bend portions or single-use handles. There exist a few major challenges in the related art. A first challenge is a related cost. A ureteroscope itself is highly costly and after a use a sterilization is also highly costly. A second challenge is about how to minimize a distal end's or a probing tip's and a catheter's diameter/perimeter. A third challenge is about how to maximize a deflection angle for maximizing accessibility. And a forth challenge is about how to keep a distal end not overheated.

### BRIEF SUMMARY OF THE PRESENT INVENTION

The present invention is about a digital flexible ureteroscope designed for a single use without any need for sterilization for another use. The present invention allows physicians to access, to visualize and to perform procedures inside patients' bodies concerned. The present invention enhances an ureteroscope's performance and reduces overall cost. Similar ideas and concepts can easily be extended to all types of endoscopes in general.

The present invention has a bend portion capable of bending 275 degrees in two directions, a distal end with multiple groups of lighting sources and imaging sensors and lenses to make a camera system transmitting real-time digital images and a working channel allowing accessing of surgery tools and irrigation. To reduce patients' uncomfortableness during the procedure, the size of the distal end in the present invention is reduced. Some embodiments illustrate groups of imaging sensors and lenses are installed on two, three or four sides of the distal ends to provide more views with a single insertion. According to different purposes of the endoscopes, lighting sources can be chosen from MicroLED/OLED/infrared LED/Laser LED/Laser diode/LED. For preventing overheating at a distal end, a few techniques for transferring heat out from the distal end area are used including applying a graphene nano-filler into various portions of an endoscope in the present invention.

### BRIEF DESCRIPTION OF THE DRAWINGS

FIG. 1 illustrates the present invention.

FIG. 2 illustrate an assembly CAD of the present invention.

FIG. 3 illustrates a rigid front end/distal end.

FIG. 4 illustrates two embodiments of the configuration of lighting source(s), a lens and imaging sensor, and a working channel.

FIG. 5A illustrates a prior art of a cross-section of a part packed in a catheter.

FIG. 5B illustrates the cross section of the part packed in the catheter.

FIG. 6 illustrates the position of lighting source(s) and the lens and imaging sensor along a z-axis of the distal end.

FIG. 7 illustrates the structure of a bend portion.

FIG. 8A illustrates the structure of a working channel connector.

FIG. 8B illustrates the structure of a front joint of bend portion.

FIG. 8C illustrates the structure of a rear joint of bend portion.

FIG. 8D illustrates the structure of a catheter sheath.

FIG. 9A illustrates the structure of a camera system.

FIG. 9B illustrates the structure of a medical wire with eight cores.

FIG. 9C illustrates the structure of the camera system in a left-side view.

FIG. 10A illustrates the structure of the working channel.

FIG. 10B illustrates the structure of a steel wire protection tube.

FIG. 10C illustrates the structure or a steel wire.

FIG. 11A illustrates the structure of an adaptor of Luer connector.

FIG. 11B illustrates the structure of the catheter.

FIG. 12A illustrates the structure of a fixing rack of steel wire protection tube.

FIG. 12B illustrates the structure of a protective cap of Luer connector.

FIG. 12C illustrates the structure of a pressure spring.

FIG. 12D illustrates the structure of an angulation control lever.

FIG. 13 illustrates the structure of a handle and the how the 8-core medical-grade wire connected to the handle.

FIG. 14 illustrates the structure of a left case.

FIG. 15 illustrates the structure of a right case.

FIG. 16A illustrates the structure of a wire wheel.

FIG. 16B illustrates the structure of a fixed plate for PCB.

FIG. 16C illustrates the structure of a wire wheel plate.

FIG. 16D illustrates the structure of a 3-way T-shaped Luer connector.

FIGS. 17A and 17B illustrate a PCB inside the handle.

FIG. 18 illustrates two models of the present inventions.

FIG. 19 illustrates an embodiment of the distal end with cameras and lighting sources installed on four sides of the distal end.

FIG. 20 illustrates an embodiment of the distal end with cameras and lighting sources installed on three sides of the distal end.

FIG. 21 illustrates an embodiment of the distal end with cameras and lighting sources installed on three sides of the distal end.

FIG. 22 illustrates an embodiment of the distal end with cameras and lighting sources installed on two sides of the distal end.

FIG. 23 illustrates an embodiment of the distal end with cameras and lighting sources installed on three sides of the distal end.

FIG. 24 illustrates a structure of the bend portion.

FIG. 25 illustrates an embodiment of the distal end that carries multiple image sensors and light sources, wherein orientations of the image sensors are adjustable via the control device/handle.

### DETAILED DESCRIPTION OF THE INVENTION

The following description with reference to exemplary and illustration drawings of the present invention will be further described in detail, but the present illustration is not intended to limit the embodiment of the present invention, any similar structure of the present invention and similar changes should be included in the scope of the present invention.

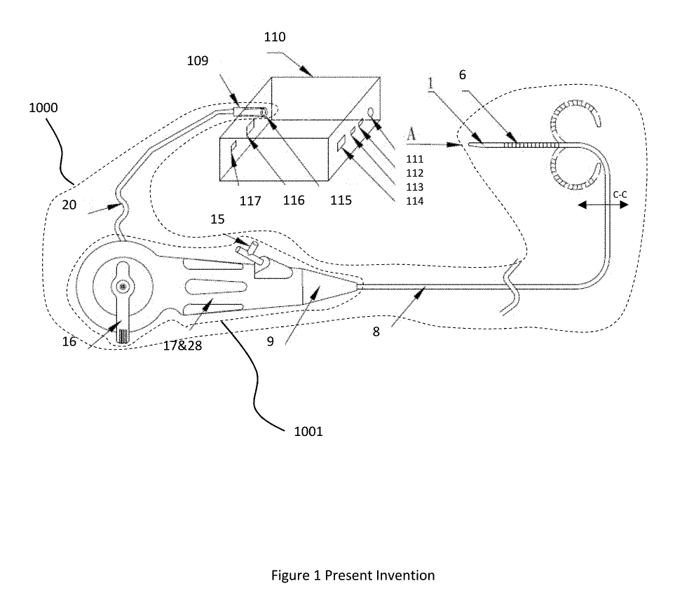

As shown in the FIG. 1, a system of the digital flexible endoscope includes a rigid front end/distal end (1), a bend portion/deflection section (6), a shaft/catheter (8), strain reliefs/catheter sheath (9), a handle/proximal portion composed of two parts (17&28) with a 3-way T-shaped Luer connector (15) and an angulation control lever (16) on it, a 8-core medical-grade wire (20), and a video processor (110) with a cable connector (109), an A/V port (111), a HDMI port (112), a USB port (113), a power supply (114), a cable plug (115), a function controller (116), and a switch (117) on it.

The Ureteroscope is used by physicians to access, to visualize, and perform procedures in the urinary tract. The ureteroscope enables delivery and use of accessories such as biopsy forceps, laser fibers, guidewires, graspers and retrieval baskets at a surgical site. The distal end (1) of the ureteroscope articulates to 275 degrees in two directions, and the distal end (1) can be rotated a total of 360 degrees by rotating the handle (17&28).

When in use, the distal end (1) is inserted into a patient's body. The bend portion (6) is controlled by the angulation control lever (16) to change the direction of the distal end (1). There are lighting source(s) (411-417, 4111-4113) and lens(es) and imaging sensor(s) (420, 4201-4203) carried on the distal end (1), so that the condition in the urethra can be captured by the lens(es) and imaging sensor(s) (420, 4201-4203), which is processed by the video processor (110) to be shown onto various display devices. Suitable tool(s) can enter the urethra via the 3-way Luer connector (15) and extend through the working channel (5) to perform at the target area.

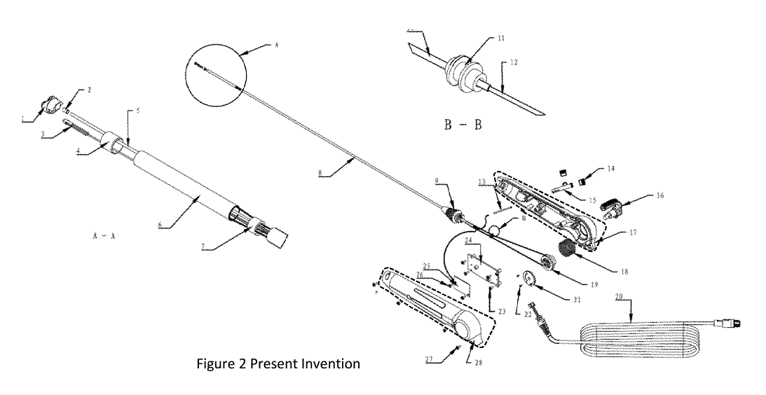

FIG. 2 is an assembly CAD of the mechanical parts of the present invention. The mechanical parts of the present invention includes the distal end (1), a working channel connector (2), a camera system (3), a front joint of bend portion (4), a working channel (5), the bend portion (6), a rear joint of bend portion (7), the catheter (8), the strain relief (9), a steel wire protection tube (10), a fixing rack o steel wire protection tube (11), a steel wire (12), an adaptor of Luer connector (13), a protective cap of Luer connector (14), the 3-way Luer connector (15), the angulation control lever (16), the right case (17), a pressure spring (18), a wire wheel 19, a 8-core medical-grade wire (20), a wire wheel plate (21), a fixed plate for PCB (24), a PCB (25), the left case (28) and screws with suitable shape and length (22, 23, 26, 27).

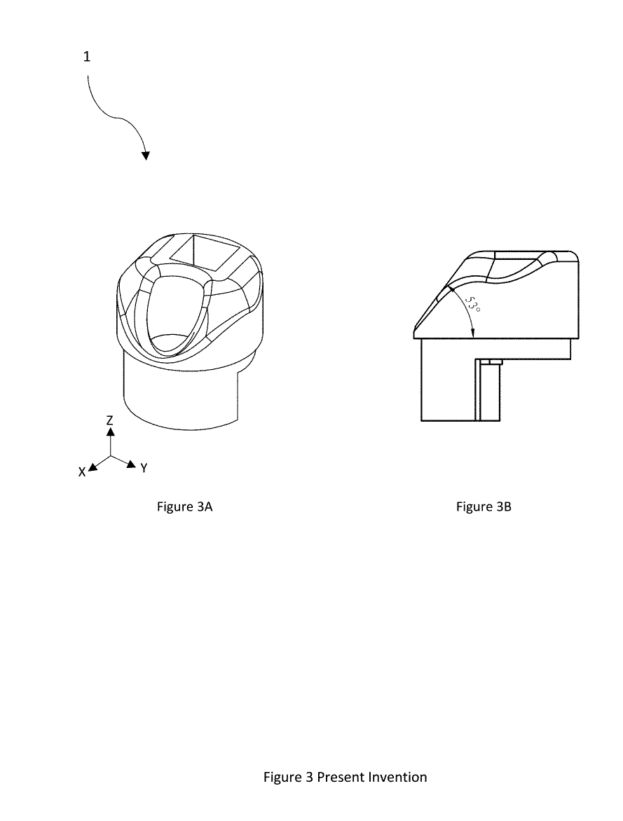

FIG. 3A is an isometric view of the rigid front end/distal end (1), and the FIG. 3B is a right view of the rigid front end/distal end (1). As shown in the FIG. 3B, the distal end (1) is designed to be ladder-shaped to reduce a cross-section area at the top of the distal end (1) which can reduce the uncomfortableness of patients.

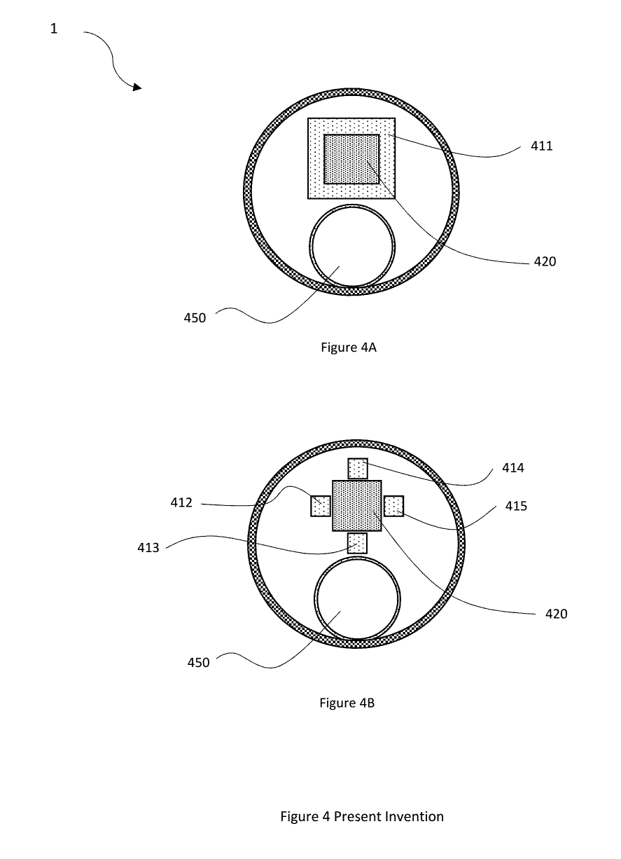

There is/are lighting source(s) (411-417, 4111-4113), the lens(es) and imaging sensor(s) (420, 4201-4203) from the camera system (3), and the working channel (5) on the surface of the distal end (1) which is covered by a shell from a top view. FIG. 4A is an embodiment of the configuration and the shape of lighting source(s) (411), the lens and imaging sensor (420) and the working channel (5). The lighting source (411) is designed to surround the lens and imaging sensor (420), and the working channel (5) is under the lens and imaging sensor (420) and the lighting source (411). The lens is fabricated on top of the imaging sensor as a single part.

FIG. 4B is an embodiment of the configuration and the shape of lighting source(s) (412-415). Four lighting sources (412-415) are arranged at four edges of the lens and imaging sensor (420), and the working channel (5) is under the lens and imaging sensor (420) and the lighting source (412-415).

The distal end (1) has a round shaped cross section, with a minimized perimeter, lens and imaging sensor (420) and an oval shaped working channel (5) next to each other in a cross-section view forming two hollow spaces inside distal end (1), in the round shaped cross section, that are filled with one or more lighting sources (411-417) in each hollow space for providing light to the imaging sensor and lens (420) yet not occupying any extra space so that a perimeter of the distal end (1) at the round shaped cross-section is minimized. And a cross-section view of the working channel (5) is an oval shape.

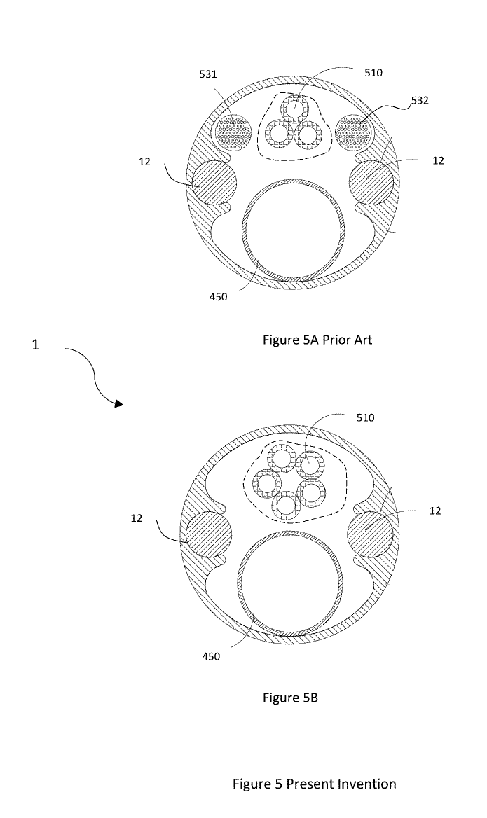

Beneath the lighting source(s) (411-417) and the camera are wires. Different from the prior art which uses fibers as lighting sources, the present invention, as shown in the FIG. 5B, has a five-wires bundle (510) include two coaxial cables and three wires. FIG. 5B is a cross-section along direction (C-C) in FIG. 1. Two coaxial cables include one for XVCLK and one for VOUT, and three wires includes one for VDD, one for LED+ and one for LED−. Each of the steel wires (12) is placed at the left and the right of the working channel (5).

The shell is manufactured through injection molding, with a mix of polymer composite material with graphene nano-filler for enhancing thermal dissipation. The shell houses the lighting source(s) (411-417, 4111-4113) and the lens and imaging sensor (420, 4201-4203).

To increase the coefficient of thermal conductivity in the distal end (1), graphene, which has a higher intrinsic thermal conductivity, is used as a nano-filler. The lighting sources (411-417, 4111-4113) are connected by graphene from the distal end (1) to middle layer of the catheter (8) which is made of 316 stainless steel weave mesh to conduct heat generated by the lighting sources (411-417, 4111-4113). Graphene nano-filler is also mixed into the material to make sheath (9) to help conducting the heat.

The lighting source(s) (411-417, 4111-4113) are selected from MicroLED/OLED/infrared LED/Laser LED/Laser diode/LED with suitable size(s), shape, intensity, and other desired parameters.

The camera system (3) is able to transmit real-time images.

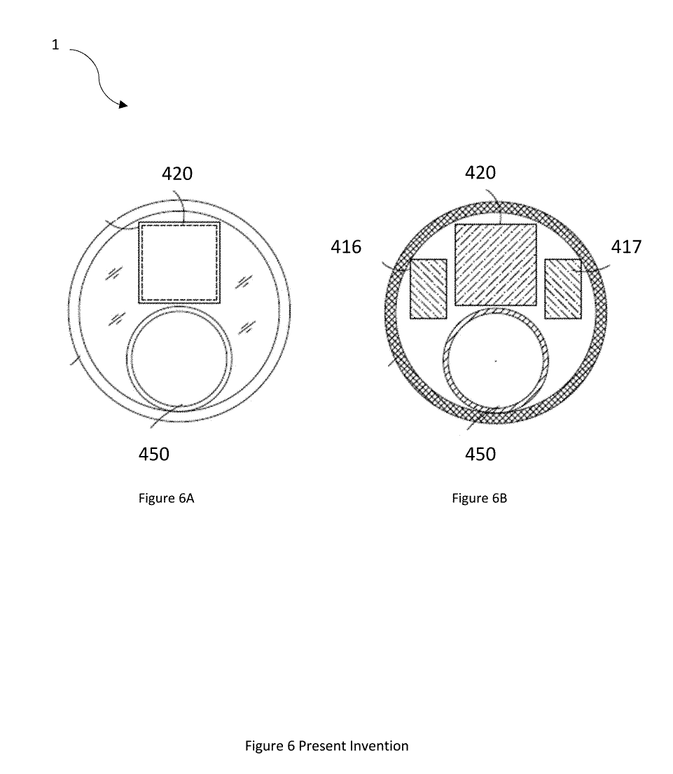

To optimize the images captured by the lens and imaging sensor (420), the lens and imaging sensor (420) is places closer to the surface of the distal end (1). As shown in the FIG. 6, if viewing along the main axis of the distal end (1), other than the working channel (5), the lens and imaging sensor (420) is seen first (FIG. 6A), and then the lighting sources (411-415) (FIG. 6B). FIG. 6B illustrates an embodiment of the two lighting sources (416-417) arranged in two sides of the lens and imaging sensor (420).

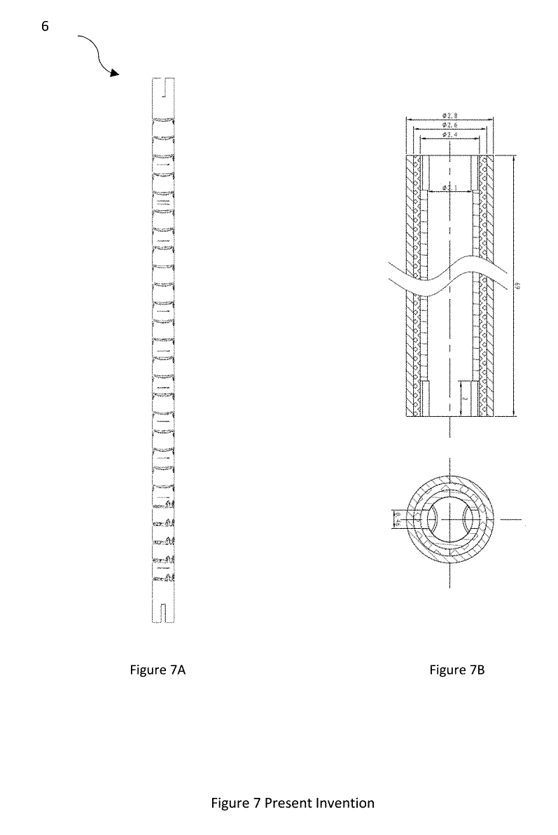

FIG. 7 shows a structure of the bend portion (6). FIG. 7A is an isometric view of the bend portion (6), and FIG. 7B is a cross-section and a top view of the bend portion (6). The bend portion (6) is able to be bent 275 degrees. The bend portion (6) is composed of three layers as shown in FIG. 7B.

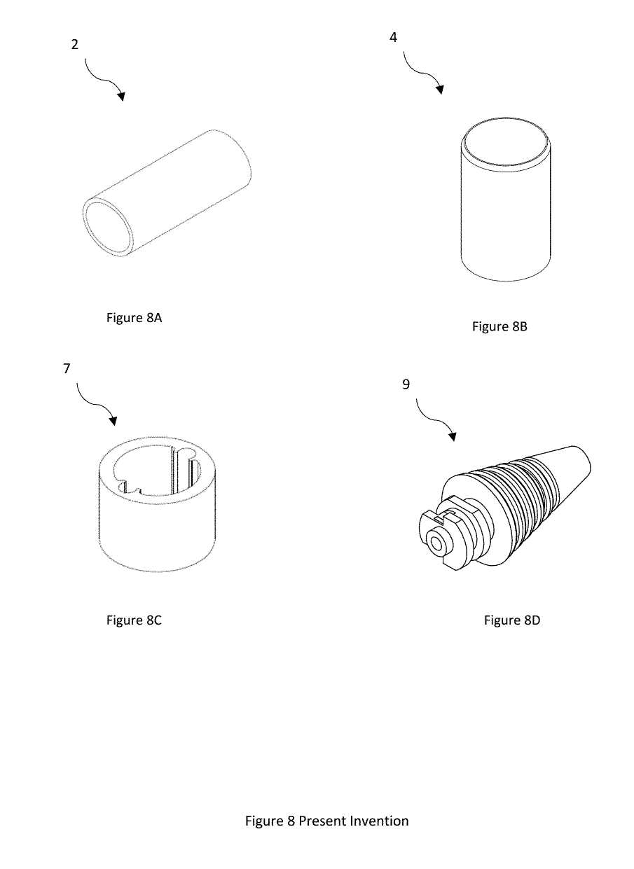

FIG. 8A is an isometric view of the working channel connector (2), FIG. 8B is an isometric view of the front joint of bend portion (4), FIG. 8C is an isometric view of the rear joint of bend portion (7) and FIG. 8D is an isometric view of the sheath (9).

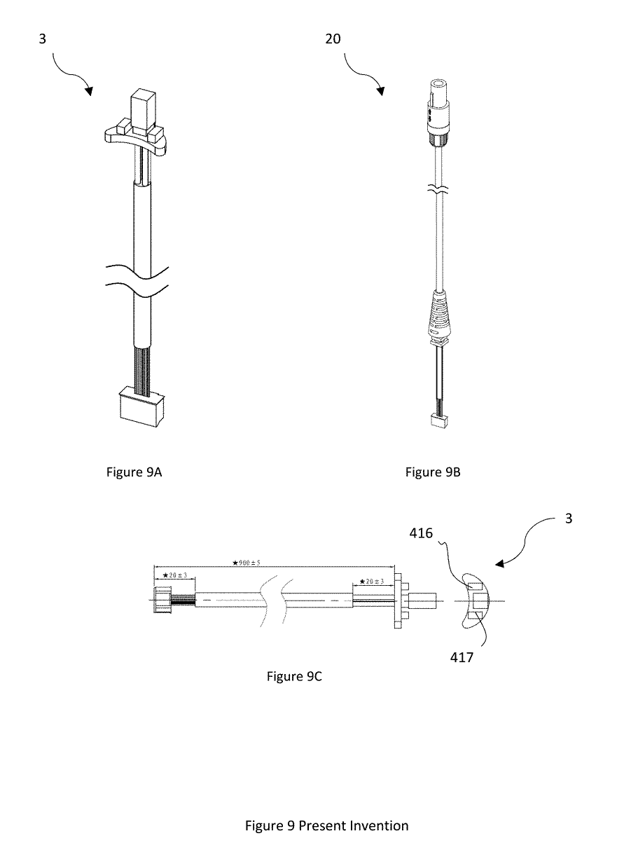

FIG. 9A is an isometric view of the camera system (3), and FIG. 9B is an isometric view of the 8-core medical-grade wire (20) which connect the video processor (110) to the handle (17&28) to conduct imaging signal. The lighting sources (416-417) are installed at a lower position than that of lens and imaging sensor (420), as shown in FIG. 9C.

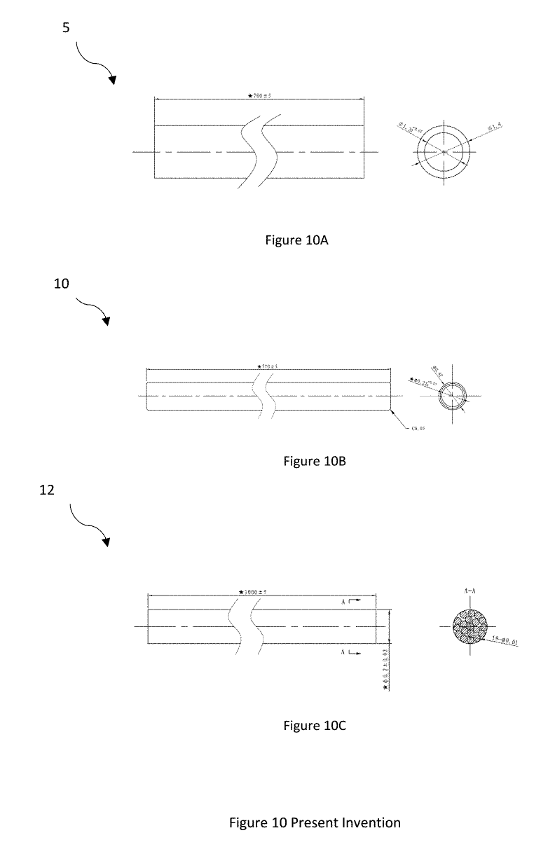

FIG. 10A is an isometric view of the working channel (5) which allows various tools to pass through. FIG. 10B is an isometric view of the steel wire protection tube (10), and the FIG. 10C is an isometric view of the steel wire (12) which is controlled by the angulation control lever (16) to manipulate the bend portion (6).

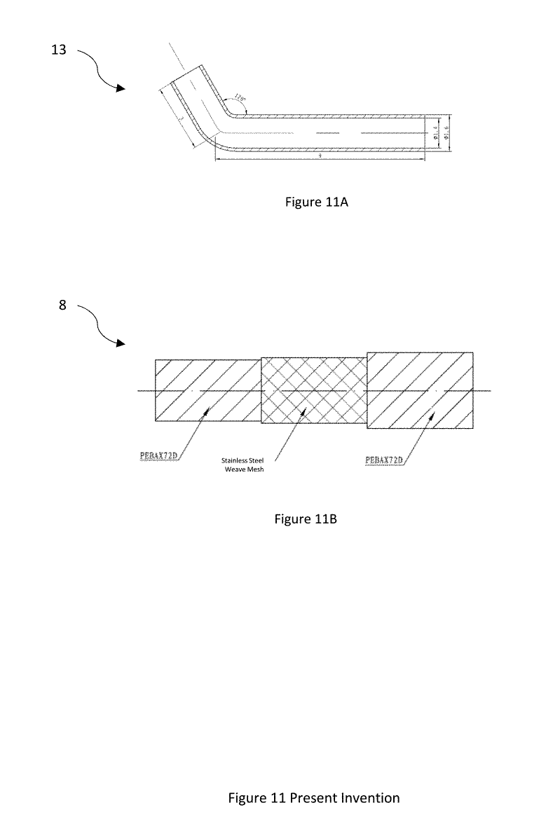

FIG. 11A is an isometric view of the adaptor of Luer connector (13), and FIG. 11B shows the three-layer catheter (8). A middle layer of the catheter (8) is stainless steel weave mesh and an inner-most layer is PEBX. An outer-layer is made by mixing graphene nano-filler into a synthetic material to enhance heat transfer conductivity of the material and to help transfer heat out from the distal end (1). With such a combination of material, the catheter (8) is able to perform as desired.

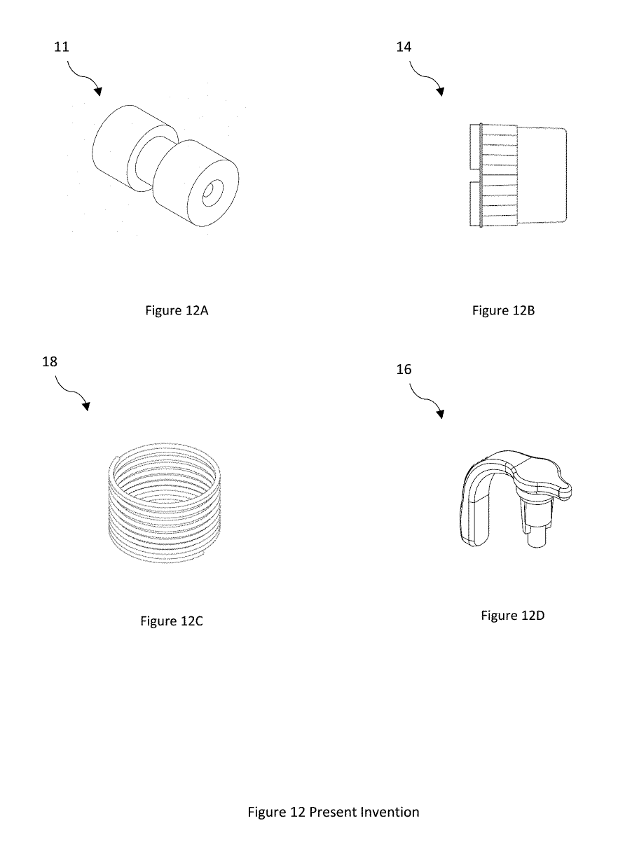

FIG. 12A is an isometric view of the fixing rack of steel wire protection tube (11), FIG. 12B is an isometric view of the protective cap of Luer connector (14), FIG. 12C is an isometric view of the pressure spring (18) and FIG. 12D is an isometric view of the angulation control lever (16). The angulation control lever (16) is installed on the handle (17&28) to control the deflection of the distal end (1).

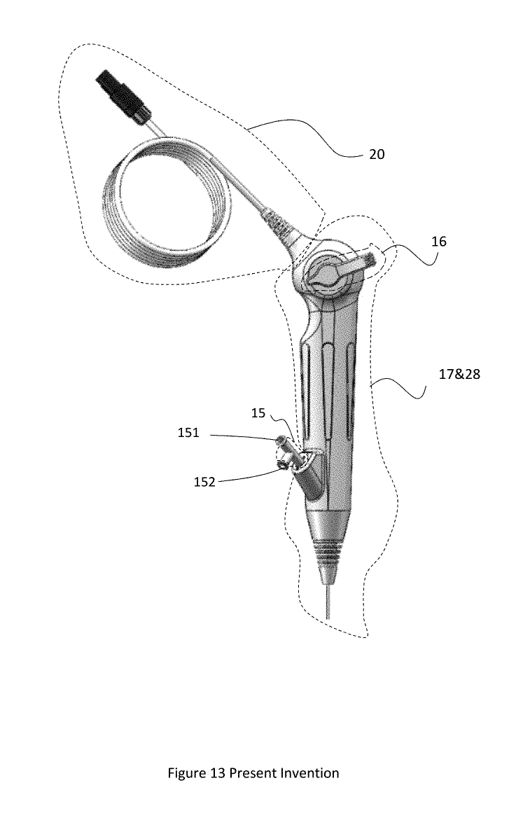

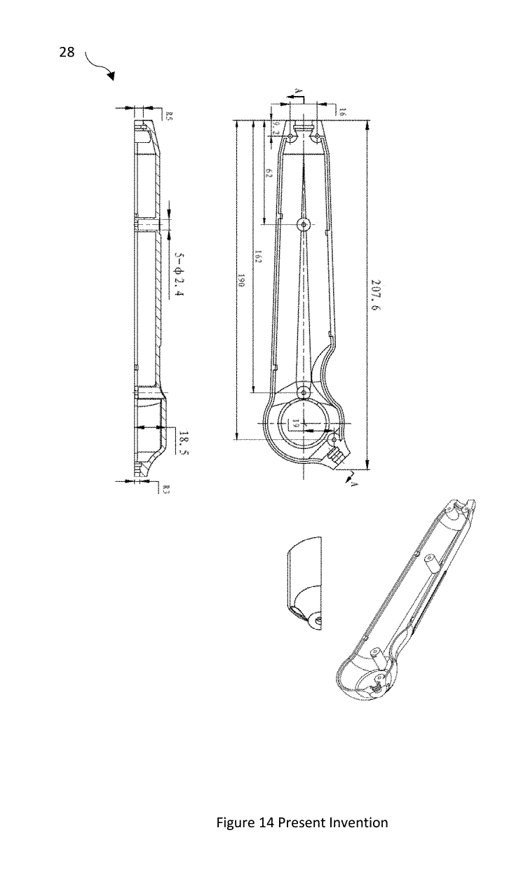

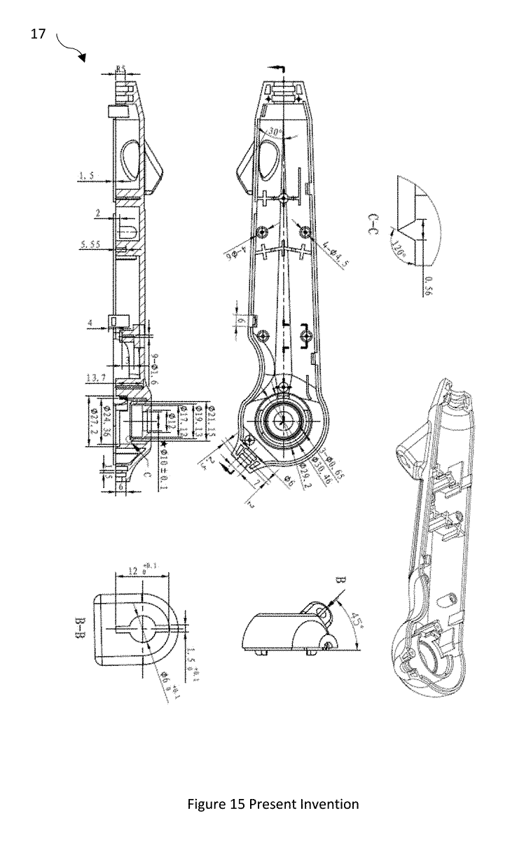

FIG. 13 is an isometric view of the handle (17&28) together with the 8-core medical-grade wire (20). The handle is fabricated from two parts—the left case (28) and the right case (17) as shown in FIG. 14 and FIG. 15. The handle (17&28) includes an articulation control lever (16) that controls articulation of the distal end (1). The handle also includes two access ports. One port accepts surgical accessories and one port provides a connection point for irrigation/contrast solution. The handle (17&28) controls the distal end (1) for insertion, withdrawal and rotation.

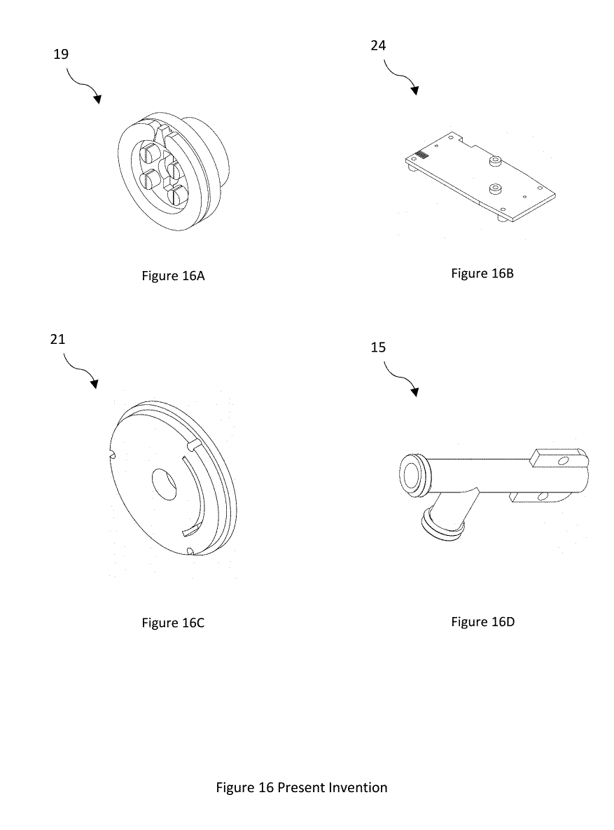

FIG. 16A is an isometric view of the wire wheel (19), FIG. 16B is the isometric view of a fixed plate for PCB (24), and FIG. 16C is the isometric view of wire wheel plate (21). FIG. 16D is the isometric view of the 3-way T-shaped Luer connector (15) with a tool inlet port and an irrigation port.

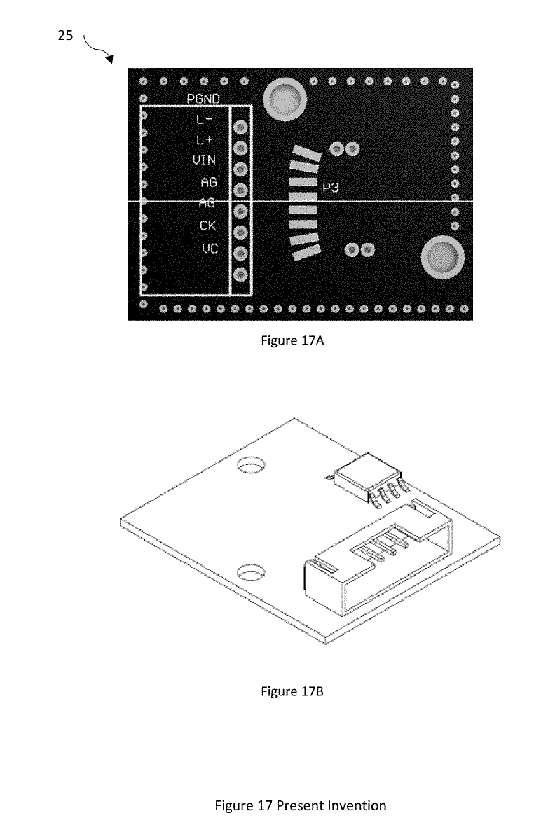

FIG. 17 shows the PCB inside the handle (25) which is used for wire bundle transition.

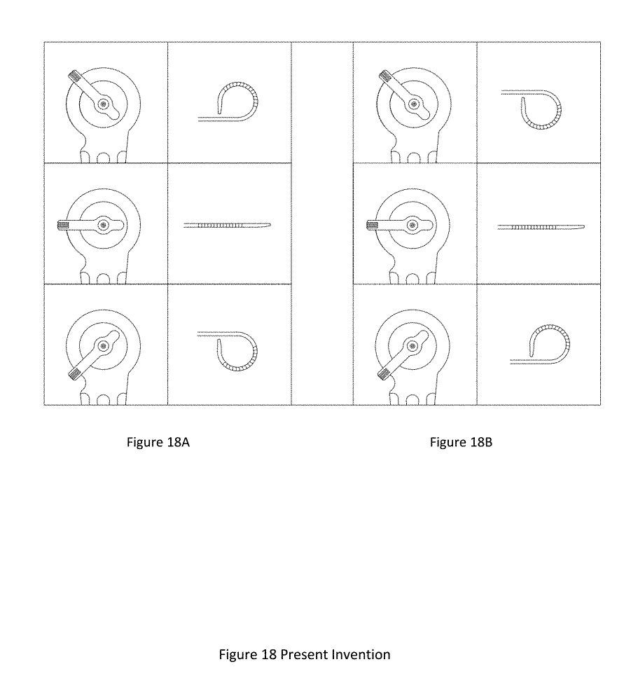

FIG. 18 shows positions of angulation control lever (16) with the corresponding bending angles of the bend portion (6). According to popular conventions adopted by many physicians, FIG. 18 illustrates two models of the present invention: XXX Model (Standard Deflection) and YYY Model (Reverse Deflection).

FIG. 18A illustrates the XXX Model, which is standard deflection. When pushing the angulation control lever (16) forward articulates the distal end (1) “up” and pushing the lever (16) back articulates the distal end (1) “down”

FIG. 18B illustrates the YYY Model, which is the reverse deflection. When pushing the angulation control lever (16) forward articulates the distal end (1) “down” and pushing the angulation control lever (16) back articulates the distal end (1) “up”.

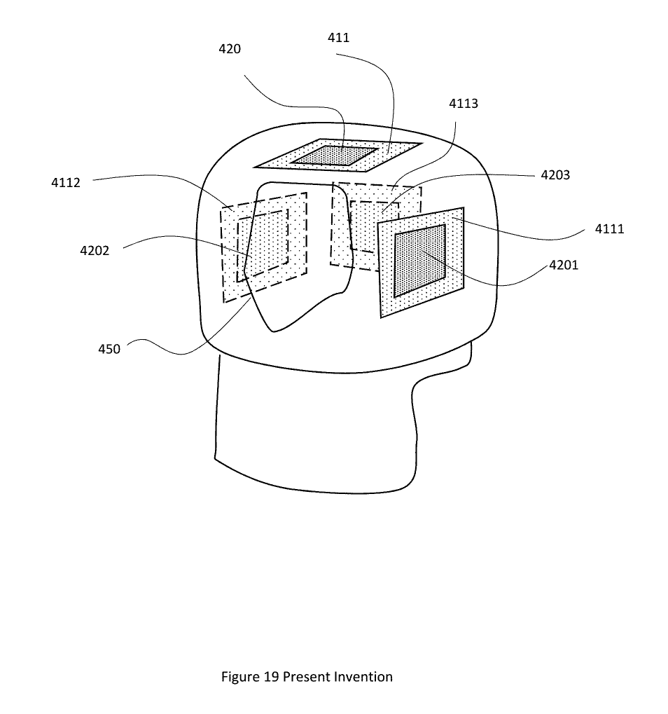

FIG. 19 illustrates an embodiment of the distal end (1) with four groups of lenses and imaging sensors (420, 4201-4203) installed on four sides of the distal end (1) with lighting sources (411, 4111-4113) surrounding them. The lenses and imaging sensors (420, 4201-4203) are able to capture images from four directions, providing a better view.

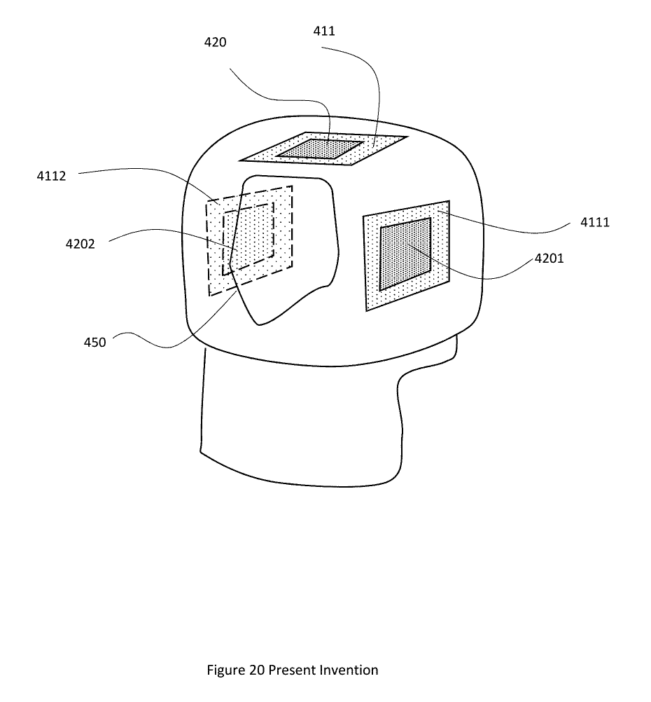

FIG. 20 illustrates an embodiment of the distal end (1) with three groups of lenses and imaging sensors (420, 4201, 4202) installed on three sides of the distal end (1) with lighting sources (411, 4111, 4112) surrounding them. The lenses and imaging sensors (420, 4201, 4202) are able to capture images from three directions, providing a better view.

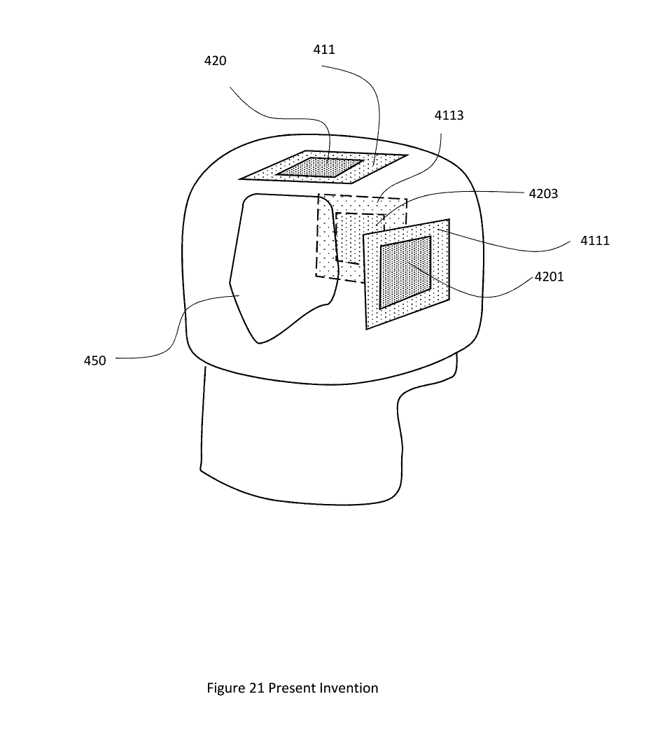

FIG. 21 illustrates an embodiment of the distal end (1) with three groups of lenses and imaging sensors (420, 4201, 4203) installed on three sides of the distal end (1) with lighting sources (411, 4111, 4113) surrounding them. The lenses and imaging sensors (420, 4201, 4203) are able to capture images from three directions, providing a better view.

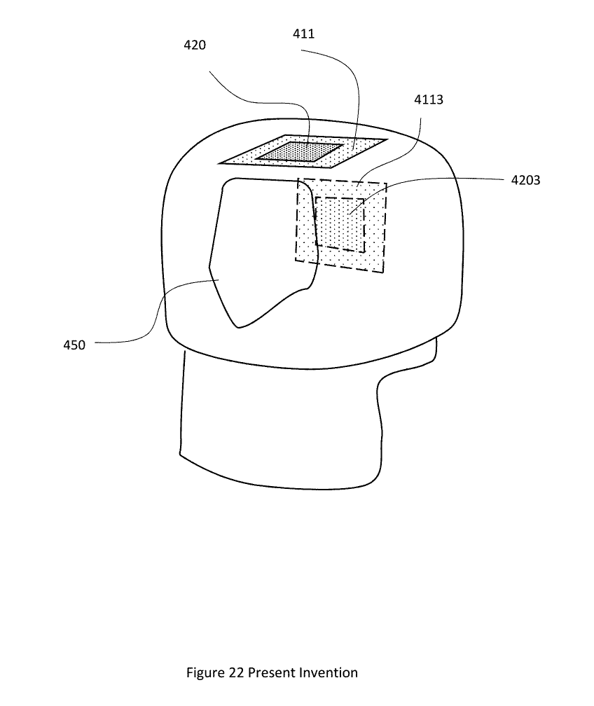

FIG. 22 illustrates an embodiment of the distal end (1) with lenses and imaging sensors (420, 4203) installed on two sides of the distal end with lighting sources (411, 4113) surrounding them. The lenses and imaging sensors (420, 4203) are able to capture images from four directions, providing a better view.

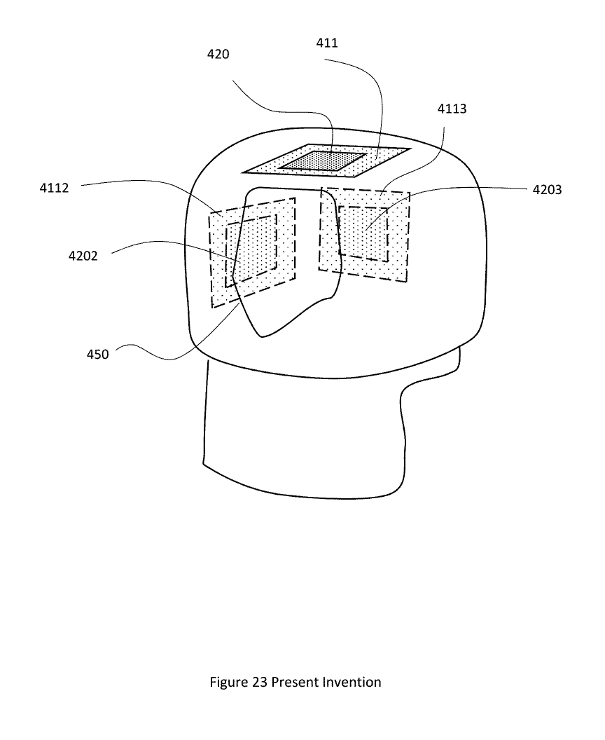

FIG. 23 illustrates an embodiment of the distal end (1) with lenses and imaging sensors (420, 4202, 4203) installed on three sides of the distal end (1) with lighting sources (411, 4112, 4113) surrounding them. The lenses and imaging sensors (420, 4202, 4203) are able to capture images from three directions, providing a better view.

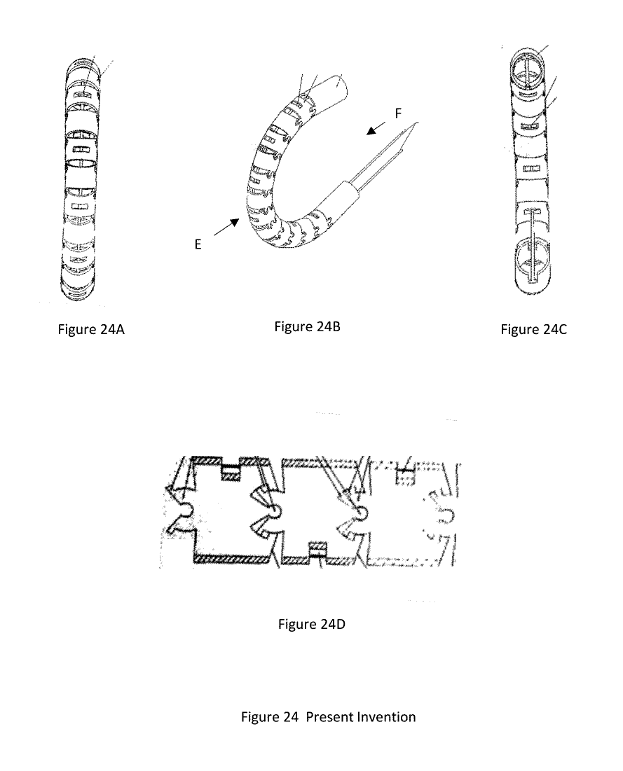

FIG. 24 illustrates the structure of the bend portion (6) with details.

FIG. 24B is an isometric view of the bend portion (6) when it is bent. FIG. 24A illustrates the structure of the bend portion (6) when it is bent viewing from direction E in FIG. 24B. FIG. 24C illustrates the structure of the bend portion (6) when it is bent viewing from direction F in FIG. 24B. As shown in FIG. 24D, the bend portion (6) is comprised of desired number of unit parts inserting into each other. The bend portion (6) has a diameter not more than 2.9 mm and a length around 690 mm if the endoscopy is for ureteroscopy.

The material to make the bend portion (6) is selected from:
i. a metal material for its high strength and its heat transfer conductivity for transferring heat out from the distal end (1);
ii. a carbon fiber material for its high strength and its heat transfer conductivity for transferring heat out from the distal end (1); and
iii. a synthetic material mixed with graphene nano-filler for enhancing its heat transfer conductivity for transferring heat out from the distal end (1).

The sheath 9, the left case (28) and the right case (17) are made from the material selected from:
a. a synthetic material; or
b. a metal material, including aluminum, for enhancing heat transfer conductivity for transferring heat out from the distal end (1) area; or
c. a graphene nano-filler, mixed with a synthetic material, for enhancing heat transfer conductivity for transferring heat out from the distal end (1) area.

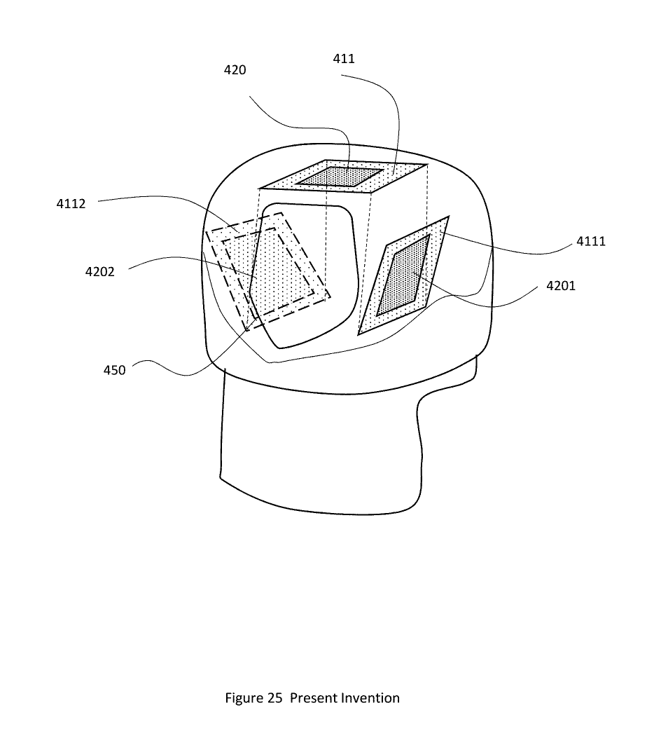

FIG. 25 illustrates an embodiment of the distal end (1) that carries three groups of imaging sensors and lenses (420, 4201, 4202) and lighting sources (411, 4111, 4112). A focal distance of a lens is a constant value, as a result, if an object is too far from the lens, cleared images cannot be captured. To get a better view when the distal end (1) is inserted into a patient's body, the distal end (1) is designed to have groups of imaging sensors and lenses (420, 4201, 4202) which orientations can be controlled.

1. An endoscope comprising
a) a video camera system (VCS) (3), inside a distal end (DE) (1) having a first end pointing to a Z direction in an X-Y-Z Cartesian coordinate system and a second end pointing to a −Z direction and sized suitable for endoscopy, comprising
i. one or more image sensors IS(s) (420, 4201-4203) facing one or more directions wherein at least a first image sensor (FIS) faces out towards the Z direction; and
ii. one or more lighting sources (LS(s)) (411-417, 4111-4113), provided by at least one from a group of MicroLED, OLED, infrared LED, Laser LED, Laser diode, and LED;
b) a bend/deflection portion (BP) (6) having a first end connected to the second end of the distal end (DE) (1) and a second end, sized suitable for endoscopy;
c) a catheter (8) having a first end connected to the second end of the bend portion (BP) (6) and a second end, sized suitable for endoscopy;
d) a control device (CD) (1001), installed to the second end of the catheter (8), for controlling the distal end (DE) (1) and accessories and for medical procedures;
e) a pair of steel wires (SW) (12) going through the control device CD (1001) and the catheter (8) for controlling the bend portion (BP) (6); and
f) a working channel (WC) (5) starting from the CD (1001) and ending at the distal end (DE) (1) through the catheter (8) and the bend portion (BP) (6) for medical treatment tools or medications accessing needed areas inside a patient's organ from the CD (1001) to the distal end (DE) (1), wherein the working channel (WC) (5) comprises an oval shaped cross section for allowing water or fluid to flow in through the working channel (WC) (5) as needed.

2. The endoscope of claim 1 wherein the VCS (3) comprises
a. alternatively a second image sensor, in addition to the first image sensor (FIS), facing out towards an X direction;
b. alternatively a third image sensor, in addition to the first image sensor (FIS) and the second image sensor (SIS), facing out towards a Y direction; and
c. alternatively a fourth image sensor, in addition to the first image sensor (FIS), the second image sensor (SIS), and the third image sensor (TIS), facing out towards a −Y direction.

3. The endoscope of claim 1 wherein the distal end (DE) (1) comprises a transparent shell (TS) as a housing for hosting the VCS (3) wherein the transparent shell (TS) comprises
a. a mixture of polymer composite material and a graphene nano-filler for enhancing thermal conductivity; and
b. a shape with smooth edges.

4. The endoscope of claim 1 wherein the LS(s) (411-417, 4111-4113) comprise/s a heat sink (HS) comprising a graphene nano-filler mixed in an adhesive material for enhancing thermal conductivity, wherein the adhesive material is used as a glue.

5. The endoscope of claim 1 wherein the LS(s) (411-417, 4111-4113) is/are located on one or more sides of an image sensor (IS) if viewed from a top for positioning the light source(s) (LS(s)) (411-417, 4111-4113).

6. The endoscope of claim 1 wherein the bend portion (BP) (6) comprises a bend part_made of chained devices tied to the pair of steel wires (SW) (12) controllable by the CD (1001) and covered by one or more layers of suitable materials for endoscopy such that the bend portion (BP) (6) is able to be bent for up to 275 degrees,
wherein the chained devices comprise
i. a metal material for its high strength and its heat transfer conductivity for transferring heat out from the distal end (DS) (1);
ii. alternatively, a carbon fiber material for its high strength and its heat transfer conductivity for transferring heat out from the distal end (DS) (1); or
iii. alternatively, a synthetic material mixed with graphene nano-filler for enhancing its heat transfer conductivity for transferring heat out from the distal end (DS) (1), and
wherein the one or more layers of suitable materials for endoscopy covering the chained devices comprise(s) an extension of the catheter (8).

7. The endoscope of claim 1 wherein the distal end (DS) (1), the bend portion (BP) (6), and the catheter have diameters not more than 2.9 mm and wherein the catheter has a length around 690 mm if the endoscopy is for ureteroscopy.

8. The endoscope of claim 1 wherein the catheter (8) comprises
a. an inner-most layer of a medical grade of material;
b. a middle layer (ML) (316) comprising a stainless steel weave mesh (SSWM) (316) wherein (ML) (316) comprises a thermal contact with the HS in the LS(s) and a thermal contact with the transparent shell (TS) in the distal end (1) for transferring heat out from the LS(s) and the IS(s); and
c. an outer-most layer of a medical grade of material.

9. The endoscope of claim 8 wherein the inner-most layer comprises a PEBX72D material and wherein the outer-most layer comprises a PEBAX72D material.

10. The endoscope of claim 8 wherein the outer-most layer alternatively comprises a graphene nano-filler, mixed with a synthetic material, for enhancing heat transfer conductivity for transferring heat out from the distal end (DS) (1) area.

11. The endoscope of claim 1 wherein the CD (1001) comprises
a. a sheath (9);
b. a left case (28);
c. a right case (17);
d. an angulation control lever (ACL) (16) for controlling a position of the distal end (DE) (1) via the SC (12);
e. a tool inlet port (TIP) (151)
f. an irrigation port (IP) (152);
g. a video signal and control cable (20);
h. a video signal and control cable connector (109) for communication with an external video processor (EVP) (110); and
i. a circuit for providing power to the IS(s) (420, 4201-4203) and the LS(s) (411-417, 4111-4113), for receiving video signals from the IS(s), and for sending video signals to the EVP (110).

12. The endoscope of claim 11 wherein angulation control lever (ACL) (16) is able to be placed at various angles for adjusting the distal end (DE) (1) at desired angles accordingly.

13. The endoscope of claim 1 wherein the distal end (DE) (1) comprises an outlet port (OP) (450) connected to the WC (5), connected to the IP (15) via the distal end (DE) (1), the bend portion (BP) (6), the catheter (8), and the control device (CD) (1001), for medical treatment tools or medications, fed at the tool inlet port (TIP) (14), accessing needed areas inside a patient's organ, wherein the oval shaped cross-section of the working channel WC (5) allows water or fluid to flow in from the irrigation port (IP) (15) to the outlet port OP (450) at the distal end (DE) (1) via the working channel (WC) (5) while a tooling catheter/tube/wire/cable/string with a round shaped cross-section occupies the working channel (WC) (5).

14. The endoscope of claim 13 wherein the outlet port (OP) is formed on the transparent shell (TS) with a slope around 53° angle from an XY plane in the XYZ Cartesian coordination system.

15. The endoscope of claim 1 wherein the distal end (DE) (1) comprises a round shaped cross section, with a minimized perimeter, that houses a square or rectangular shaped image sensor IS (420, 4201-4203) and an oval shaped working channel (WC) (5) next to each other in a cross-section view forming two hollow spaces inside distal end (DE) (1), in the round shaped cross section, that are filled with one or more lighting sources (LS(s)) (411-417, 4111-4113) in each hollow space for providing light to the first image sensor (FIS) (420) yet not occupying any extra space so that a perimeter of the distal end (DE) (1) at the round shaped cross-section is minimized.

16. The endoscope of claim 11 wherein the sheath (9), the left case (28), and the right case (17) comprise one or more from:
a. a synthetic material;
b. a metal material, including aluminum, for enhancing heat transfer conductivity for transferring heat out from the distal end (DS) (1) area; and
c. a graphene nano-filler, mixed with a synthetic material, for enhancing heat transfer conductivity for transferring heat out from the distal end (DS) (1) area.

17. The endoscope of claim 11 wherein the sheath (9) comprises one or more from:
a. a synthetic material;
b. a metal material, including aluminum, for enhancing heat transfer conductivity for transferring heat out from the distal end (DS) (1) area; and
c. a graphene nano-filler, mixed with a synthetic material, for enhancing heat transfer conductivity for transferring heat out from the distal end (DS) (1) area.

18. The endoscope of claim 11 wherein the left case (28) and right case (17) the comprise one or more from:
a. a synthetic material;
b. a metal material, including aluminum, for enhancing heat transfer conductivity for transferring heat out from the distal end (DS) (1) area; and
c. a graphene nano-filler, mixed with a synthetic material, for enhancing heat transfer conductivity for transferring heat out from the distal end (DS) (1) area.

19. The endoscope of claim 1 wherein one or more image sensor(s)′ (4200, 4201-4203) orientation(s) is/are adjustable via the control device (CD) (1001).

20. The endoscope of claim 8 wherein the middle layer (ML) (316) alternatively comprises a carbon fiber weave mesh (CFWM) for enhancing its strength and heat transfer conductivity for transferring heat out from the distal end (DS) (1) area and for reducing a thickness of the middle layer (ML) (316).
